(19) United States
(12) Patent Application Publication
(10) Pub. No.: US 2005/0014674 A1
(43) Pub. Date: Jan. 20, 2005

(54) Cosmetic compositions for making up and/or caring for skin

(75) Inventors: Anne Liechty, Paris (FR); Euriel Clavel, Paris (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 10/853,337

(22) Filed: May 26, 2004

Related U.S. Application Data
(60) Provisional application No. 60/485,392, filed on Jul. 9, 2003.

(30) Foreign Application Priority Data
May 28, 2003 (FR) 03 06491

(51) Int. Cl. A61K 7/46

(52) U.S. Cl. 512/1

(57) ABSTRACT

The present invention relates to a cosmetic composition for caring for and/or making up the skin and/or the lips, comprising at least one liquid fatty phase, the said fatty phase containing at least one lipodispersible film-forming polymer and polyethylene particles with a number-average size of less than or equal to 50 μm.

The present invention relates to a cosmetic composition for making up and/or caring for the skin, especially the face.

This composition may especially be in the form of products cast in stick or dish form, for instance lipsticks or lip balms, cast foundations, concealer products, eyeshadows or makeup rouges, in more or less fluid paste or cream form, for instance fluid foundations or lipsticks, eyeliners, antisun compositions or skin-colouring compositions, or alternatively body makeup compositions.

Makeup or care products for the human skin or lips, for instance foundations or lipsticks, generally contain a fatty phase based on oil(s) and/or wax(es), pigments and/or fillers and optionally additives, for instance cosmetic or dermatological active agents.

More particularly, the compositions under consideration according to the invention can constitute a makeup product, for example for the face, of which the long-term staying power of the matt effect of the makeup is prolonged, i.e. the visual degradation of which over time is significantly reduced.

By definition, a matting product is a product that prevents the skin from shining and that unifies the complexion. It is already known practice to use compositions with a matting effect. However, these compositions are not entirely satisfactory. They do not generally make it possible to provide a sustained matt effect over time and their application needs to be freshened regularly, especially beyond four hours of exposure, in order to ensure the expected effect.

There is thus a need for a cosmetic composition that can give a matt makeup effect with good staying power over time in terms of the matt effect.

The inventors have found, unexpectedly, that it is possible to overcome the abovementioned drawback by incorporating, into the fatty phase of this type of composition, polyethylene particles in combination with at least one lipodispersible film-forming compound.

Consequently, according to one of its aspects, the present invention relates to a cosmetic composition for caring for and/or making up the skin and/or the lips, comprising at least one liquid fatty phase, the said fatty phase containing at least one film-forming polymer and polyethylene particles with a number-average size of less than or equal to 50 μm.

According to another of its aspects, a subject of the invention is also a cosmetic care and/or makeup process comprising at least one step of application of a composition according to the invention to the skin and/or the lips.

According to another of its aspects, the invention is also directed towards the use of polyethylene particles according to the invention in a matting cosmetic composition, to obtain a matt effect that is sustained over time. In particular, these polyethylene particles may be used in combination with at least one film-forming polymer chosen from liposoluble or lipodispersible film-forming polymers and mixtures thereof.

Polyethylene Particles

The polyethylene particles according to the invention have a number-average size of less than or equal to 50 μm, in particular less than or equal to 30 μm and more particularly less than or equal to 20 μm.

The term “number-average size” denotes the dimension given by the statistical particle size distribution to half of the population, referred to as the D50.

The polyethylene particles may be in various forms and in particular may be substantially spherical.

For the purposes of the present invention, the term “polyethylene particles” covers any particle derived from the homopolymerization and/or copolymerization of the ethylene monomer. These particles may, where appropriate, include in their composition compounds generally in trace amount, resulting from their preparation process.

According to one particular variant of the invention, the polyethylene particles may be particles comprising at least one ethylene/acrylic acid copolymer, and in particular may consist of ethylene/acrylic acid copolymer(s), for instance the particles sold under the trade name Flobeads® by the company SUMITOMO.

The polyethylene particles may, where appropriate, be surface-coated, at least partially, with dyestuffs, especially pigments and/or dyes.

In one composition according to the invention, the polyethylene particles may be present in a content ranging from 0.1% to 20% by weight, especially from 0.5% to 15% by weight and in particular from 1% to 10% by weight relative to the total weight of the composition.

Film-forming Polymer

The composition according to the invention comprises at least one film-forming polymer chosen from liposoluble or lipodispersible film-forming polymers and mixtures thereof.

Liposoluble Polymer

The liposoluble polymers may be of any chemical nature and especially include:

a) liposoluble, amorphous homopolymers and copolymers of olefins, of cycloolefins, of butadiene, of isoprene, of styrene, of vinyl ethers, esters or amides, or of (meth)acrylic acid esters or amides comprising a linear, branched or cyclic C₄-C₅₀ alkyl group. The liposoluble homopolymers and copolymers may be chosen from those obtained from monomers chosen from the group consisting of isooctyl (meth)acrylate, isononyl (meth)acrylate, 2-ethylhexyl (meth)acrylate, lauryl (meth)acrylate, isopentyl (meth)-acrylate, n-butyl (meth)acrylate, isobutyl (meth)acrylate, tert-butyl (meth)acrylate, tridecyl (meth)acrylate and stearyl (meth)acrylate, or mixtures thereof. Examples that will be mentioned include the alkyl acrylate/cycloalkyl acrylate copolymer sold by PHOENIX CHEM under the name GIOVAREZ AC-5099 ML.

Liposoluble film-forming polymers that may also be mentioned include vinylpyrrolidone (VP) copolymers and especially copolymers of vinylpyrrolidone and of a C₂-C₄₀ and in particular C₃ to C₂₀ alkene. As examples of VP copolymers that may be used in the invention, mention may be made of copolymers of VP/vinyl acetate, VP/ethyl methacrylate, VP/ethyl methacrylate/methacrylic acid, VP/eicosene, VP/hexadecene, VP/triacontene, VP/styrene, VP/acrylic acid/lauryl methacrylate and butylated polyvinylpyrrolidone (PVP).

Particular liposoluble copolymers that may also be mentioned include:

i) acrylic-silicone grafted polymers containing a silicone skeleton and acrylic grafts or containing an acrylic skeleton and silicone grafts, such as the products sold under the names “SA 70.5”, “SA 70” and “VS 80” by 3M and described especially in patents U.S. Pat. No. 5,725,882, U.S. Pat. No. 5,209,924, U.S. Pat. No. 4,972,037, U.S. Pat. No. 4,981,903, U.S. Pat. No. 4,981,902, U.S. Pat. No. 5,468,477, U.S. Pat. No. 5,219,560, EP 0 388 582, U.S. Pat. No. 5,032,460 and WO 93/23009,

ii) liposoluble polymers belonging to one of the classes described above and bearing fluoro groups, in particular those described in patent U.S. Pat. No. 5,948,393 and the alkyl (meth)acrylate/perfluoroalkyl (meth)acrylate copolymers described in patents EP 0 815 836 and U.S. Pat. No. 5,849,318,

iii) polymers or copolymers resulting from the polymerization or copolymerization of an ethylenic monomer, comprising one or more ethylenic bonds, which are preferably conjugated (or diene). This or these agent(s) is (are) in particular vinyl, acrylic or methacrylic copolymers, which may be in block form and especially of diblock or triblock type, or even of multiblock or starburst type.

The ethylenic film-forming polymer may especially comprise a styrene (S) block, an alkylstyrene (AS) block, an ethylene/butylene (EB) block, an ethylene/propylene (EP) block, a butadiene (B) block, an isoprene (I) block, an acrylate (A) block, a methacrylate (MA) block or a combination of these blocks.

In particular, the film-forming polymer used may be a copolymer comprising at least one styrene block. Most particularly, a triblock copolymer may be used and in particular those of the polystyrene/polyisoprene or polystyrene/polybutadiene type, such as those sold under the name “LUVITOL HSB” by BASF, and those of the polystyrene/copoly(ethylene-propylene) type or alternatively of the polystyrene/copoly(ethylene-butylene) type, such as those sold under the brand name “KRATON” by Shell Chemical Co. or Gelled Permethyl 99A by Penreco. Styrene-methacrylate copolymers may also be used.

As film-forming polymers that may be used in the composition of the invention, examples that may also be mentioned include KRATON G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton G1750X (EP) multiarm, Kraton G1765X (EP) multiarm, Kraton D-1101 (SBS), Kraton D-1102 (SBS), Kraton D-1107 (SiS), Gelled Permethyl 99A-750, Gelled Permethyl 99A-753-58 (blend of triblock and of starburst block polymer), Gelled Permethyl 99A-753-59 (blend of triblock and of starburst block polymer), Versagel 5970 and Versagel 5960 from Penreco (blend of triblock and of starburst polymer in isododecane), and OS 129880, OS 129881 and OS 84383 from Lubrizol (styrene-methacrylate copolymer);

b) amorphous liposoluble polycondensates, in particular those containing no groups that give hydrogen interaction, especially polyesters containing C₄-C₅₀ alkyl side chains or polyesters resulting from the condensation of fatty acid dimers, or alternatively polyesters comprising a silicone segment in block, graft or terminal group form, which are solid at room temperature, for example as defined in patent application FR-A-2831430;

c) amorphous liposoluble polysaccharides comprising alkyl (ether or ester) side chains, in particular alkylcelluloses comprising a linear or branched, saturated or unsaturated C₁-C₈ alkyl radical such as ethylcellulose and propylcellulose.

In general, the film-forming liposoluble polymers of the invention may have a weight-average molecular weight ranging from 1000 to 500 000 and especially from 2000 to 250 000, and a glass transition temperature ranging from −100° C. to +300° C., especially from −50° C. to +100° C. and in particular from −10° C. to +90° C.

Lipodispersible Polymer

The lipodispersible polymer is generally present in the form of a stable dispersion of particles, which are generally spherical and solid, in the liquid fatty phase. These dispersions may especially be in the form of polymer nanoparticles in dispersion. These nanoparticles may have a size ranging from 5 to 600 nm and especially ranging from 50 to 250 nm.

The dispersed polymer particles that may be used in the composition according to the invention may have a weight-average molecular weight ranging from about 2000 to 10 000 000.

The polymer may have a glass transition temperature ranging from −100° C. to +300° C., especially from −10° C. to +50° C. and more particularly less than or equal to about +40° C.

The polymer used in the present invention in the form of particles dispersed in the fatty phase may be of any nature. Thus, a free-radical polymer, a polycondensate, or even a polymer of natural origin, and blends thereof, may be used. The polymer may be chosen by a person skilled in the art on the basis of its properties.

It is more particularly a “film-forming” polymer, i.e. a polymer capable of forming, alone or in combination with a plasticizer, an isolable film.

Illustrations of film-forming polymers that may be mentioned include acrylic or vinyl free-radical homopolymers and copolymers, especially those with a glass transition temperature Tg of less than or equal to about +40° C. and in particular ranging from −10° C. to +30° C., and blends thereof.

The expression “free-radical polymer” means a polymer obtained by polymerization of monomers containing unsaturation, especially ethylenic unsaturation, each monomer being capable of homopolymerizing (unlike polycondensates). The free-radical polymers may especially be vinyl polymers or copolymers, especially acrylic polymers.

The vinyl polymers may result from the polymerization of ethylenically unsaturated monomers containing at least one acid group and/or esters of these acidic monomers and/or amides of these acids.

Monomers bearing an acidic group that may be used include α,β-ethylenic unsaturated carboxylic acids such as acrylic acid, methacrylic acid, crotonic acid, maleic acid and itaconic acid. (Meth)acrylic acid and crotonic acid are preferably used, and more preferably (meth)acrylic acid.

The esters of acidic monomers are advantageously chosen from (meth)acrylic acid esters (also known as (meth)acrylates), for instance (meth)acrylates of an alkyl, in particular of a C₁-C₂₀ and preferably C₁-C₈ alkyl, (meth)acrylates of an aryl, in particular of a C₆-C₁₀ aryl, (meth)acrylates of a hydroxyalkyl, in particular of a C₂-C₆ hydroxyalkyl.

Alkyl (meth)acrylates that may be mentioned include methyl, ethyl, butyl, isobutyl, 2-ethylhexyl and lauryl (meth)acrylate.

Hydroxyalkyl (meth)acrylates that may be mentioned include hydroxyethyl (meth)acrylate and 2-hydroxypropyl (meth)acrylate.

Aryl (meth)acrylates that may be mentioned include benzyl or phenyl acrylate.

Particularly advantageous (meth)acrylic acid esters are alkyl (meth)acrylates.

Free-radical polymers that may especially be used include copolymers of (meth)acrylic acid and of an alkyl (meth)acrylate, especially of a C₁-C₄ alkyl. More particularly, methyl acrylates optionally copolymerized with acrylic acid, such as copolymers of poly(methyl acrylate/acrylic acid) type, may be used.

Amides of the acidic monomers that may be mentioned include (meth)acrylamides, and especially N-alkyl(meth)acrylamides, in particular of a C₂-C₁₂ alkyl, such as N-ethylacrylamide, N-t-butylacrylamide and N-octylacrylamide, and N-di(C₁-C₄)alkyl(meth)acrylamides.

The vinyl polymers may also result from the polymerization of ethylenically unsaturated monomers containing at least one amine group, in free form or in partially or totally neutralized form, or alternatively in partially or totally quaternized form. Such monomers may be, for example, chosen from dimethylaminoethyl (meth)acrylate, dimethylaminoethylmethacrylamide, vinylamine, vinylpyridine or diallyldimethylammonium chloride.

The vinyl polymers may also result in the homopolymerization or copolymerization of at least one monomer chosen from vinyl esters and styrene monomers. In particular, these monomers may be polymerized with acidic monomers and/or esters thereof and/or amides thereof, such as those mentioned above.

Examples of vinyl esters that may be mentioned include vinyl acetate, vinyl propionate, vinyl neodecanoate, vinyl pivalate, vinyl benzoate and vinyl t-butylbenzoate.

Styrene monomers that may be mentioned include styrene and α-methylstyrene.

The list of monomers given is not limiting and it is possible to use any monomer known to those skilled in the art falling within the categories of acrylic and vinyl monomers (including monomers modified with a silicone chain).

As other vinyl monomers that may be used, mention may also be made of:
N-vinylpyrrolidone, vinylcaprolactam, vinyl-N-(C₁-C₆)alkylpyrroles, vinyloxazoles, vinylthiazoles, vinylpyrimidines and vinylimidazoles, and
olefins such as ethylene, propylene, butylene, isoprene and butadiene.

The vinyl polymer may be crosslinked with the aid of difinctional monomers especially comprising at least two ethylenic unsaturations, such as ethylene glycol dimethacrylate or diallyl phthalate.

In a nonlimiting manner, the polymers of the invention may be chosen from the following polymers or copolymers: polyurethanes, polyurethane-acrylics, polyureas, polyurea-polyurethanes, polyester-polyurethanes, polyether-poly-urethanes polyesters, polyesteramides, alkyd fatty-chain polyesters; acrylic-silicone copolymers; polyacrylamides; silicone polymers, fluoro polymers, and mixtures thereof.

According to one particular variant of the invention, the lipodispersible polymers may be surface-stabilized with at least one liposoluble polymer, for instance those described below or those described in patent application EP 1 002 528.

The film-forming polymer dispersion may be prepared as described in document EP-A-749 747 or in document EP 1 002 528. More specifically, the polymerization is performed in dispersion, i.e. by precipitation of the polymer as it is formed, with protection of the formed particles with a stabilizer. Stabilizers are especially described in EP 749 747.

In general, the composition according to the invention may comprise from 0.1% to 25% by weight, especially from 1% to 20% by weight and in particular from 5% to 16% by weight of film-forming polymer and especially of lipodispersible film-forming polymer (expressed as solids), relative to the total weight of the composition.

The composition according to the invention may comprise at least one auxiliary film-forming agent that promotes the formation of a film with the film-forming polymer. Such a film-forming agent may be chosen from any compound known to those skilled in the art as being capable of satisfying the desired function, and may be chosen especially from plasticizers and coalescers.

The coalescers or plasticizers that may be used in the invention are especially those mentioned in document FR-A-2 782 917.

Liquid Fatty Phase

The liquid fatty phase of the composition may comprise one or more cosmetically or dermatologically acceptable and generally physiologically acceptable oils.

Thus, it may comprise at least one oil, which may be chosen especially from carbon-based, hydrocarbon-based, fluoro-based and/or silicone-based oils of mineral, animal, plant or synthetic origin, alone or as a mixture, provided that they form a uniform, stable mixture and that they are compatible with the intended use.

The term “liquid fatty phase” means any non-aqueous medium that is liquid at room temperature (25° C.) and atmospheric pressure. This fatty phase may contain a volatile liquid fatty phase and/or a non-volatile fatty phase.

The term “volatile fatty phase” means any non-aqueous medium capable of evaporating from the skin or the lips in less than one hour. This volatile phase especially comprises oils with a vapour pressure, at room temperature and atmospheric pressure, ranging from 10⁻³ to 300 mmHg (0.13 Pa to 40 000 Pa).

The liquid fatty phase according to the invention may comprise at least one compound chosen from hydrocarbon-based oils such as liquid paraffin or liquid petroleum jelly, mink oil, turtle oil, soybean oil, perhydrosqualene, sweet almond oil, calophyllum oil, palm oil, grapeseed oil, sesame seed oil, maize oil, parleam oil, arara oil, rapeseed oil, sunflower oil, cottonseed oil, apricot oil, castor oil, avocado oil, jojoba oil, olive oil or cereal germ oil; esters of lanolic acid, of oleic acid, of lauric acid or of stearic acid; fatty esters such as isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl triisostearate or diglyceryl triisostearate; higher fatty acids such as myristic acid, palmitic acid, stearic acid, behenic acid, oleic acid, linoleic acid, linolenic acid or isostearic acid; higher fatty alcohols such as cetanol, stearyl alcohol, oleyl alcohol, linoleyl alcohol, linolenyl alcohol, isostearyl alcohol or octyldodecanol; silicone oils such as polydimethylsiloxanes (PDMS), which are optionally phenylated, such as phenyl trimethicones, or optionally substituted with aliphatic and/or aromatic groups, which are optionally fluorinated, or optionally substituted with functional groups such as hydroxyl, thiol and/or amine groups; polysiloxanes modified with fatty acids, fatty alcohols or polyoxyalkylenes, fluorosilicones and perfluoro oils.

Advantageously, one or more oils that are volatile at room temperature may be used.

They may be hydrocarbon-based oils or silicone oils and may optionally comprise alkyl or alkoxy groups, which are pendent or at the end of a silicone chain.

As volatile oils that may be used in the invention, mention may be made of linear or cyclic silicones containing from 2 to 7 silicon atoms, these silicones optionally comprising alkyl or alkoxy groups containing from 1 to 10 carbon atoms, especially such as octamethylcyclotetrasiloxane, decamethylcyclopentasiloxane, hexadecamethylcyclohexasiloxane, heptamethylhexyltrisiloxane and heptamethyloctyltrisiloxane, and also C₈-C₁₆ isoparaffins such as the ISOPAR® and PERMETHYL® products, and especially isododecane.

The total liquid fatty phase of the composition may represent from 5% to 97% by weight and especially from 20% to 85% by weight relative to the total weight of the composition. The non-volatile part may represent from 0.1% to 80% by weight and especially from 1% to 50% by weight relative to the total weight of the composition.

Solid Fatty Substances

The composition may also comprise at least one fatty substance that is pasty or solid at room temperature, chosen, for example, from waxes and gums, and mixtures thereof.

For the purposes of the present invention, the term “wax” means a lipophilic fatty compound that is solid at room temperature (25° C.), with a reversible solid/liquid change of state, having a melting point of greater than 30° C., which may be up to 200° C., a hardness of greater than 0.5 MPa, and an anisotropic crystal organization in the solid state. By bringing the wax to its melting point, it is possible to make it miscible with the oils and to form a microscopically homogeneous mixture, but on returning the temperature of the mixture to room temperature, recrystallization of the wax in the oils of the mixture is obtained.

The hardness of the wax is determined by measuring the compression force, measured at 20° C. using a texturometer sold under the name TA-XT2i by the company Rheo, equipped with a stainless-steel cylinder 2 mm in diameter travelling at a measuring speed of 0.1 mm/s and penetrating into the wax to a penetration depth of 0.3 mm. To perform the hardness measurement, the wax is melted at a temperature equal to the melting point of the wax +20° C. The molten wax is poured into a container 30 mm in diameter and 20 mm deep. The wax is recrystallized at room temperature (25° C.) for 24 hours and is then stored for at least one hour at 20° C. before performing the hardness measurement. The value of the hardness is the measured compression force divided by the area of the texturometer cylinder in contact with the wax.

For the purposes of the present patent application, the waxes may be hydrocarbon-based waxes, silicone waxes and/or fluoro waxes and may optionally comprise ester or hydroxyl functions. They may especially be of natural or synthetic origin. Non-limiting illustrations of waxes that may especially be mentioned include optionally modified beeswax, carnauba wax, candelilla wax, ouricury wax, Japan wax, cork fibre wax or sugarcane wax, ceresin, paraffin wax, lignite wax, microcrystalline waxes, lanolin wax, montan wax, ozokerites, hydrogenated oils, for instance hydrogenated jojoba oil or ethylene copolymerization oil, waxes obtained by Fischer-Tropsch synthesis, fatty acid esters and glycerides that are solid at 45° C., silicone waxes, for instance alkyl, alkoxy and/or esters of poly(di)methylsiloxane that are solid at 45° C., containing from 10 to 45 carbon atoms, and certain fatty acids, for instance stearic acid, myristic acid or behenic acid, and mixtures thereof.

The composition according to the invention may comprise at least one wax in a content ranging from 0.1% to 20% by weight, especially from 2% to 15% by weight and in particular from 3% to 12% by weight relative to the total weight of the composition.

Aqueous Phase

The composition according to the invention may also comprise an aqueous phase containing water. The water may be a floral water such as cornflower water and/or a mineral water such as eau de VITTEL, eau de LUCAS or eau de LA ROCHE POSAY and/or a spring water.

The aqueous phase may also comprise solvents other than water, for instance primary alcohols such as ethanol and isopropanol, glycols such as glycerol, propylene glycol, butylene glycol, dipropylene glycol, diethylene glycol, glycol ethers, such as C₁-C₄ alkyl ethers of mono-, di- or tripropylene glycol, or mono-, di- or triethylene glycol, and mixtures thereof.

The aqueous phase may also comprise stabilizers, for example sodium chloride, magnesium dichloride and magnesium sulphate.

The aqueous phase may also comprise any water-soluble or water-dispersible compound that is compatible with an aqueous phase, such as gelling agents, film-forming polymers, thickeners and surfactants, and mixtures thereof.

The aqueous phase, especially water, may especially be present in the composition according to the invention in a content ranging from 2% to 75% by weight and in particular from 5% to 50% by weight relative to the total weight of the composition.

The composition may be present in the form of an oil-in-water (O/W) or water-in-oil (W/O) emulsion. It may also be present in anhydrous form.

When the composition according to the invention is in the form of an emulsion, it may also comprise a surfactant or a mixture of surfactants whose HLB (hydrophilic/lipophilic balance) value is generally suited to the nature of the emulsion to be stabilized.

As surfactants that may be used in the invention, which are suitable for producing a W/O emulsion, mention may be made of those with an HLB value of less than 7 and especially fatty acid esters of polyols, for instance glyceryl or sorbitol mono-, di-, tri- or sesquioleates or stearates, and glyceryl or polyethylene glycol laurates; alkyl or alkoxy dimethicone copolyols containing an alkyl or alkoxy chain that is pendent or at the end of a silicone skeleton, containing, for example, from 6 to 22 carbon atoms. As surfactants that may be used in the invention to produce an O/W emulsion, mention may be made of those with an HLB value of greater than 7, for instance fatty acid esters of polyethylene glycol (polyethylene glycol monostearate or monolaurate); polyoxyethylenated fatty acid esters (stearate or oleate) of sorbitol; polyoxyethylenated alkyl (lauryl, cetyl, stearyl or octyl) ethers and dimethicone copolyols. In general, any amphoteric ionic (cationic or anionic) surfactant and any nonionic surfactant, which are well known to those skilled in the art, may be used.

The surfactant may be present in the composition in a content ranging from 0.3% to 10% by weight and especially from 1% to 5% by weight relative to the total weight of the composition.

The composition according to the invention may also comprise at least one additional pulverulent compound, i.e. a compound other than the polyethylenes considered according to the invention. The additional pulverulent compound may be chosen from fillers, pulverulent dyestuffs such as pigments and nacres, and mixtures thereof.

The term “fillers” should be understood as meaning white or colourless, mineral or synthetic, lamellar or non-lamellar particles.

These fillers may be chosen from talc, mica, silica, kaolin, poly-β-alanine powder, powders of lauroyllysine, starch, boron nitride, hollow polymer microspheres such as those of polyvinylidene chloride/acrylonitrile, such as Expancel® (Nobel Industrie), acrylic polymer particles, especially of acrylic acid copolymer, for instance Polytrap® (Dow Corning), polyurethane powders, silicone resin microbeads (for example Tospearls® from Toshiba), precipitated calcium carbonate, dicalcium phosphate, magnesium carbonate and magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms and preferably from 12 to 18 carbon atoms, for example zinc stearate, magnesium stearate or lithium stearate, zinc laurate and magnesium myristate, and mixtures thereof.

These fillers may or may not be surface-treated, especially to make them lipophilic.

These fillers may be present in the composition in a content ranging from 0.1% to 30% by weight and especially from 0.1% to 10% by weight relative to the total weight of the composition.

For the purposes of the present invention, the term “pigments” is intended to denote white or colourless, mineral or organic particles that are insoluble in the liquid organic phase, which are intended to colour and/or opacify the composition.

The pigments may be mineral and/or organic pigments. Pigments that may be used include metal oxides, for instance iron oxides (especially yellow, red, brown or black iron oxides), titanium dioxides, cerium oxide, zirconium oxide and chromium oxide; manganese violet, ultramarine blue, Prussian blue, ferric blue, bismuth oxychloride, natural mother-of-pearl, mica coated with titanium or with bismuth oxychloride, coloured nacreous pigments such as titanium mica with iron oxides, titanium mica especially with ferric blue or chromium oxide, titanium mica with an organic pigment of the abovementioned type and nacreous pigments based on bismuth oxychloride, and mixtures thereof.

Iron oxide or titanium dioxide pigments are used in particular.

The pigments may be treated, where appropriate, with a hydrophobic agent to make them compatible with the fatty phase of the composition.

Such hydrophobic-treated pigments are especially described in patent application EP-A-1 086 683.

For the purposes of the present invention, the term “nacres” is intended to denote iridescent particles, produced especially by certain molluscs in their shell or else synthesized, which are insoluble in the medium of the composition.

The pulverulent dyestuffs may be present in a content ranging from 0.5% to 30% by weight, especially ranging from 5% to 20% by weight and in particular ranging from 6% to 15% by weight relative to the total weight of the composition.

The composition may also comprise at least one lipophilic dye and/or at least one hydrophilic dye.

For the purposes of the present invention, the term “dye” is intended to denote compounds, generally organic compounds, which are soluble in the fatty substances such as the oils or in an aqueous-alcoholic phase.

Among the liposoluble dyes that may be used according to the invention, mention may be made of Sudan Red, D&C Red No. 17, D&C Green No. 6, β-carotene, soybean oil, Sudan Brown, D&C Yellow No. 11, D&C Violet No. 2, D&C Orange No. 5, quinoline yellow, annatto and bromo acids.

The composition according to the invention may also contain one or more adjuvants that are common in cosmetics, such as hydrophilic or lipophilic gelling agents and/or thickeners; moisturizers; emollients; hydrophilic or lipophilic active agents; free-radical scavengers; sequestering agents; antioxidants; preserving agents; basifying or acidifying agents; fragrances; and mixtures thereof.

As active agents that may be used in the composition according to the invention, mention may be made, for example, of moisturizers such as protein hydrolysates and polyols, for instance glycerol, glycols, for instance polyethylene glycols, and sugar derivatives; natural extracts; antiinflammatory agents; procyannidol oligomers; vitamins such as vitamin A (retinol), vitamin E (tocopherol), vitamin C (ascorbic acid), vitamin B5 (panthenol), vitamin B3 (niacinamide), derivatives of these vitamins (especially esters) and mixtures thereof; urea; caffeine; salicylic acid and its derivatives; α-hydroxy acids such as lactic acid and glycolic acid and derivatives thereof; retinoids such as carotenoids and vitamin A derivatives; sunscreens; hydrocortisone; melatonin; algal, fungal, plant, yeast or bacterial extracts; enzymes; steroids; antibacterial active agents, for instance 2,4,4′-trichloro-2′-hydroxydiphenyl ether (or triclosan), 3,4,4′-trichloro-carbanilide (or triclocarban) and the acids indicated above, and especially salicylic acid and its derivatives; tensioning agents; and mixtures thereof.

The composition according to the invention may also comprise at least one sunscreen (or UV-screening agent). This agent may be chosen from organic screening agents and physical sunblocks, and mixtures thereof.

As chemical sunscreens that may be used in the composition of the invention, the composition of the invention may comprise any UVA- and UVB-screening agents that may be used in cosmetics.

Needless to say, a person skilled in the art will take care to select the optional adjuvant(s) added to the composition according to the invention and to adjust the amounts thereof such that the advantageous properties intrinsically associated with the composition in accordance with the invention are not, or are not substantially, adversely affected by the envisaged addition.

The examples that follow are given, with no limiting nature, to illustrate the invention.

### EXAMPLE 1

### Preparation of a Dispersion of a Lipodispersible Film-forming Polymer

A dispersion of a non-crosslinked copolymer of methyl acrylate and of acrylic acid in an 85/15 ratio, in isododecane, is prepared according to the method of Example 7 of document EP-A-749 747. A dispersion of poly(methyl acrylate/acrylic acid) particles surface-stabilized in isododecane with a polystyrene/copoly(ethylene-propylene) diblock copolymer sold under the name KRATON G1701 (Shell), having a solids content of 22.6% by weight, a mean particle size of 175 nm (polydispersity: 0.05) and a Tg of 20° C., is thus obtained. This copolymer can form a film at room temperature (25° C.).

### EXAMPLE 2

A foundation in the form of a water-in-oil emulsion having the composition below was prepared:

| | |
|---|---|
| Oily phase: | |
| Dispersion of lipodispersible film-forming polymer of | 55.52 g |
| Example 1 | |
| Isododecane gelled with Kraton (triblock and radial) | 4.48 g |
| sold under the trade name Versagel M5950 by the company | |
| Penreco | |
| Isododecane | 1.97 g |
| Blend of polydimethylsiloxane containing a-ω oxyethylene | 0.90 g |
| oxypropylene groups (DP: 100-58/42) | |
| and of cyclopentasiloxane | |
| (85/15) sold under the trade name “Abil EM 97” | |
| by the company Goldschmidt | |
| Cyclopentadimethylsiloxane | 4.00 g |
| Nylon 12 powder sold under the trade name Orgasol 2002 | 3.00 g |
| Extra D Nat. Cos. by the company Atochem | |
| Ethylene/acrylic copolymer powder (particle size: 10 μm) | 4.00 g |
| sold under the name Flobeads ® EA-209 by the company | |
| Sumitomo | |
| Mixture of disteardimonium hectorite, cyclopentasiloxane | 3.53 g |
| and ethanol (10/85/5) (Bentone Gel V5-5V from Elementis | |
| Specialities) | |
| Isostearic acid mono-di-glycerides esterified with succinic | 0.30 g |
| acid, non-stabilized, sold under the trade name | |
| “Inwitor 780 K” by the company Sasol | |
| Iron oxides coated with aluminium stearoylglutamate | 3.21 g |
| Titanium oxide coated with aluminium stearoylglutamate | 6.79 g |
| Aqueous phase: | |
| Glycerol | 3.00 g |
| Magnesium sulfate | 0.70 g |
| Preserving agents qs | |
| Water qs | 100.00 g |

The emulsion is prepared by mixing together, at room temperature, the constituents of the oily phase and then adding the aqueous phase, prepared beforehand, with stirring.

This foundation applies easily to the skin and has a good feeling of creaminess, and the makeup result obtained affords a soft deposit and the matt effect at the time of application is satisfactory and persists for well beyond four hours.

1. Cosmetic composition for caring for and/or making up the skin and/or the lips, comprising at least one liquid fatty phase, the said fatty phase containing at least one lipodispersible film-forming polymer and polyethylene particles with a number-average size of less than or equal to 50 μm.

2. Composition according to claim 1, characterized in that the polyethylene particles have a number-average size of less than or equal to 30 μm and especially less than or equal to 20 μm.

3. Composition according to claim 1 or 2, characterized in that the polyethylene particles comprise at least one ethylene/acrylic acid copolymer and consist especially of ethylene/acrylic acid copolymer(s).

4. Composition according to any one of the preceding claims, characterized in that it comprises from 0.1% to 20% by weight, especially from 0.5% to 15% by weight and in particular from 1% to 10% by weight of polyethylene particles relative to the total weight of the composition.

5. Composition according to any one of the preceding claims, characterized in that the film-forming polymer is chosen from liposoluble and lipodispersible film-forming polymers, and mixtures thereof.

6. Composition according to claim 5, characterized in that the liposoluble film-forming polymer is chosen from liposoluble, amorphous homopolymers and copolymers of olefins, of cycloolefins, of butadiene, of isoprene, of styrene, of vinyl ethers, esters or amides, or of (meth)acrylic acid esters or amides comprising a linear, branched or cyclic C₄₋₅₀ alkyl group; amorphous liposoluble polycondensates, especially polyesters containing C₄₋₅₀ alkyl side chains or polyesters resulting from the condensation of fatty acid dimers; liposoluble amorphous polysaccharides comprising alkyl (ether or ester) side chains; vinylpyrrolidone (VP) copolymers; acrylic silicone grafted polymers containing an acrylic skeleton and silicone grafts or containing a silicone skeleton and acrylic grafts; and blends thereof.

7. Composition according to claim 5 or 6, characterized in that the film-forming polymer comprises at least one triblock copolymer and in particular a copolymer of the polystyrene/polyisoprene or polystyrene/polybutadiene type or alternatively of the polystyrene/copoly(ethylene-butylene) or polystyrene/copoly(ethylene-propylene) type.

8. Composition according to any one of claims 5 to 7, characterized in that the lipodispersible film-forming polymer is chosen from polyurethanes, polyurethane-acrylics, polyureas, polyurea-polyurethanes, polyester-polyurethanes, polyether-poly-urethanes, polyesters, polyesteramides, fatty-chain polyesters, alkyds, acrylic and/or vinyl polymers or copolymers, acrylic-silicone copolymers, polyacrylamides, silicone polymers and fluoro polymers, and blends thereof.

9. Composition according to claim 8, characterized in that the lipodispersible film-forming polymer is chosen from acrylic or vinyl free-radical homopolymers and copolymers, especially having a glass transition temperature (Tg) of less than or equal to +40° C. and in particular ranging from −10 to +30° C., and blends thereof.

10. Composition according to any one of claims 8 or 9, characterized in that the lipodispersible polymer is an acrylic polymer of poly(methyl acrylate/acrylic acid) type.

11. Composition according to any one of claims 1 to 7, characterized in that the said lipodispersible film-forming polymer is surface-stabilized with at least one liposoluble film-forming polymer, in particular as defined in claims 6 or 7.

12. Composition according to any one of the preceding claims, characterized in that it comprises at least from 0.1% to 25% by weight, especially from 1% to 20% by weight and in particular from 5% to 16% by weight of film-forming polymer (expressed as solids) relative to the total weight of the composition.

13. Composition according to any one of the preceding claims, characterized in that the liquid fatty phase comprises at least one oil chosen from carbon-based, hydrocarbon-based, fluoro-based and/or silicone-based oils of plant, mineral, animal or synthetic origin, and mixtures thereof.

14. Composition according to any one of the preceding claims, characterized in that the liquid fatty phase comprises at least one compound chosen from liquid paraffin or liquid petroleum jelly, mink oil, turtle oil, soybean oil, perhydrosqualene, sweet almond oil, calophyllum leaf oil, palm oil, parleam oil, grapeseed oil, sesame seed oil, maize oil, rapeseed oil, sunflower oil, cottonseed oil, apricot oil, castor oil, avocado oil, jojoba oil, olive oil or cereal germ oil; esters of lanolic acid, of oleic acid, of lauric acid or of stearic acid; fatty esters such as isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl triisostearate or diglyceryl triisostearate; higher fatty acids such as myristic acid, palmitic acid, stearic acid, behenic acid, oleic acid, linoleic acid, linolenic acid or isostearic acid; higher fatty alcohols such as cetanol, stearyl alcohol, oleyl alcohol, linoleyl alcohol, linolenyl alcohol, isostearyl alcohol or octyldodecanol; silicone oils such as polydimethylsiloxanes (PDMS), which are optionally phenylated, such as phenyl trimethicones, or optionally substituted with aliphatic and/or aromatic groups, or optionally substituted with functional groups such as hydroxyl, thiol and/or amine groups; polysiloxanes modified with fatty acids, fatty alcohols or polyoxyalkylenes, fluoro oils and perfluoro oils; volatile oils such as octamethylcyclotetrasiloxane, decamethylcyclopentasiloxane, hexamethylcyclohexasiloxane, heptamethylhexyltrisiloxane, heptamethyloctyltrisiloxane or C₈-C₁₆ isoparaffins, and isododecane.

15. Composition according to any one of the preceding claims, characterized in that the liquid fatty phase is present in a content ranging from 5% to 97% by weight and especially from 20% to 85% by weight relative to the total weight of the composition.

16. Composition according to any one of the preceding claims, characterized in that it also comprises at least one fatty substance that is pasty or solid at room temperature, chosen from waxes and gums, and mixtures thereof.

17. Composition according to any one of the preceding claims, characterized in that it is in the form of a water-in-oil or oil-in-water emulsion.

18. Composition according to any one of the preceding claims, characterized in that it is anhydrous.

19. Composition according to any one of the preceding claims, characterized in that it comprises at least one additional pulverulent compound chosen from fillers, pigments and nacres, and mixtures thereof.

20. Composition according to any one of the preceding claims, characterized in that it is in the form of a makeup composition.

21. Composition according to any one of the preceding claims, characterized in that it is in the form of a foundation, a concealer product, an eyeshadow, a makeup rouge, an eyeliner, an antisun composition, a skin-colouring composition or a body makeup composition.

22. Composition according to claim 21, characterized in that it is a foundation.

23. Cosmetic process for caring for and/or making up the skin and/or the lips, comprising at least one step of applying to the skin and/or the lips a composition according to any one of the preceding claims.

24. Use of polyethylene particles as defined according to any one of claims 1 to 4, in a matting cosmetic composition to obtain a long-lasting matt effect.

25. Use according to claim 24, the said polyethylene particles being combined with at least one film-forming polymer chosen from liposoluble or lipodispersible film-forming polymers, and mixtures thereof.
